(19) United States
(12) Patent Application Publication
(10) Pub. No.: US 2019/0311791 A1
(43) Pub. Date: Oct. 10, 2019

(54) SYSTEM AND METHOD FOR PATIENT-CENTRIC UNIVERSAL HEALTH RECORDING AND PAYMENT

(71) Applicant: Healthcard LLC, Plantation, FL (US)

(72) Inventor: Miguel St. Paul, North Bay Village, FL (US)

(73) Assignee: Healthcard LLC, Plantation, FL (US)

(21) Appl. No.: 15/944,867

(22) Filed: Apr. 4, 2018

(51) Int. Cl. G16H 10/65; G06Q 20/34

(52) U.S. Cl. CPC G16H 10/65; G06Q 20/34

(57) ABSTRACT

A patient-centric universal health recording and payment system can include a synchronized server having synchronized patient information accessible and controllable by a patient, a plurality of electronic medical record databases selectively synchronized with the synchronized server, and a universal user interface for presenting the synchronized patient information including information from all accessed providers. The system can further include a plurality of third party inputs to the synchronized server selectively providing information regarding the patient, a plurality of patient sourced inputs to the synchronized server selectively providing information about the patient, and a patient input defining accessibility among and between the plurality of electronic medical record databases, a source of the plurality of third party inputs, and a source of the plurality of patient sourced inputs. The system can further include a patient health card providing patient authentication or patient identification, and funding for payments of health care services.

### FIELD OF THE DISCLOSURE

The present disclosure generally relates to systems and methods for providing patient-centric universal health recording and payment including a synchronized server having synchronized patient information accessible and controllable by a patient.

### BACKGROUND

While the technology has been developed to provide the capability of storing medical records electronically, the use and implementation of electronic medical records has not developed significantly beyond the traditional physician-controlled medical record system based on paper medical records.

Some systems disclose a network-based system and method for ordering and reporting cumulative results of medical tests from a single provider. The physician computer and lab site computer are interconnected by a computer network. The physician computer receives a physician or user request for ordering a test, causes a test request message to be sent to the lab site computer, causes a request for statistical data to be sent to the network, and receives statistical data from the network. The lab site computer is programmed to receive a test request message and to cause a test results message or a test status message to be sent to the physician computer. The system also includes a patient database computer which generates longitudinal medical reports, and performs test ordering functions, real time results reporting, and intelligent physician alerting and decision support functions, as appropriate in response to requests from other computers in the system. No patient access to or control of their medical records is disclosed nor is an integrated means of payment provided. Other systems repeatedly lack patient access and control of their own medical records and an integrated means of payment.

The primary difference between prior art medical information systems and the present invention is the ability of the patient to access and control their medical data and to further make payments to third parties. Medical providers can include pharmacies, medical laboratories, clinical practices, doctors, hospitals, nurses, and other health care providers.

As the Information Age has allowed more and more personal data to be collected, stored, used, and often even sold, privacy concerns of patients have assumed more importance. Many of the prior art electronic medical record systems have included mechanisms to provide some amount of privacy for patients by limiting access to medical records to authorized medical personnel, but have not allowed patients to decide which medical personnel will be authorized to receive or information or to enter additional data.

### BRIEF DESCRIPTION OF DRAWINGS

FIG. 1 is a flow chart of a method of a patient-centric universal health recording and payment in accordance with an embodiment;

FIG. 2 is a high level block diagram of a system using the method of FIG. 1 in accordance with an embodiment;

FIG. 3 is another high level block diagram of a system using the method of FIG. 1 in accordance with an embodiment;

FIG. 4 is a block diagram of a system illustrating interactions among an a synchronized database, an Electronic Medical Record (EMR), and a patient in accordance with an embodiment;

FIG. 5 is a block diagram illustrating a flow of interactions in accordance with an embodiment;

FIG. 6 is another block diagram illustrating a flow of interactions in accordance with an embodiment;

FIG. 7 is another block diagram illustrating a flow of interactions in accordance with an embodiment;

FIG. 8 is another block diagram illustrating a flow of interactions in accordance with an embodiment;

FIG. 9 is another block diagram illustrating a flow of interactions in accordance with an embodiment;

FIG. 10 is another block diagram illustrating a flow of interactions in accordance with an embodiment;

FIG. 11 is a block diagram of a system illustrating interactions among an a synchronized database, an Electronic Medical Record (EMR), a telemedicine service provider and a patient card in accordance with an embodiment;

FIG. 12 is another block diagram illustrating a flow of interactions in accordance with an embodiment;

FIG. 13 is another block diagram illustrating a flow of interactions in accordance with an embodiment;

FIG. 14 is another block diagram illustrating a flow of interactions in accordance with an embodiment;

FIG. 15 is a block diagram illustrating a synchronized server interacting with a service provider such as a hospital in accordance with an embodiment;

FIGS. 16A, 16B, and 16C illustrate graphical user interfaces on a client device in accordance with an embodiment;

FIGS. 17A, 17B, and 17C illustrate graphical user interfaces on a client device reflecting a patient survey in accordance with an embodiment;

FIG. 18 is a flow chart of a method of creating and utilizing healthcard currency in accordance with an embodiment;

FIG. 19 illustrates a system for patient-centric universal health recording and payment in accordance with an embodiment.

### DETAILED DESCRIPTION

There is a disparity between the amount of information collected by the electronic health record (EHR) technology and the number of physicians and patients who utilize and manipulate the data. Being that is uncommon for hospitals or providers of clinical settings to utilize the same EHR system which poses an interoperability conundrum. The information contained in physician notes, progress notes, and radiology reports provides a comprehensive view of the treatment of a particular patient. The aggregation of such documents across a larger population of patients provides the foundation for analysis of quality of care, treatment protocols, patient outcomes, drug effectiveness, and the effectiveness and durability of medical devices. The aggregation and managed patient control of such information has been lacking. Furthermore, a means of integrating payment or incentivizing healthful activities has also been lacking. In some embodiments, the “HealthCard” or patient identity and payment card is what the healthcare system has been lacking in the form of a universal platform, which enables clinicians and other medical providers to sort and organize medical history data into meaningful information making diagnosing and test ordering more efficient.

Often times a patient's coverage information is difficult to view and typically the patient's appropriate insurance card is unavailable when needed. With the hundreds of medical insurance plans for a patient to choose from, keeping track of the necessary billing information is a job in and of itself.

Medical Coverage Information should be easy to find, and even easier to share. Every year new coverage plans are available, and often your previous billing information will not be acceptable anywhere current insurance information is needed. With HelathCard keeping your payment information stored safely on a Smart Card, checking out of a pharmacy does not need to be painful anymore.

Healthcard has a robust middle ware software with alleviates the interoperability issues within an EMR system meeting all protocol standards HL7 along with FHIR. Helath Level-7 is a set of international standards for transfer of clinical and administrative data between software application used by various healthcare providers. FHIR stands for Fast Healthcare Interoperability Resources which is a standard describing data formats and elements and an application programming interface for exchanging electronic health records. Patients are provided with a Master Patient Idea which enables a comprehensive robust view of patients records for providers to utilize. As we make the transition from fee for service to more value based care, it is imperative that such integration take place.

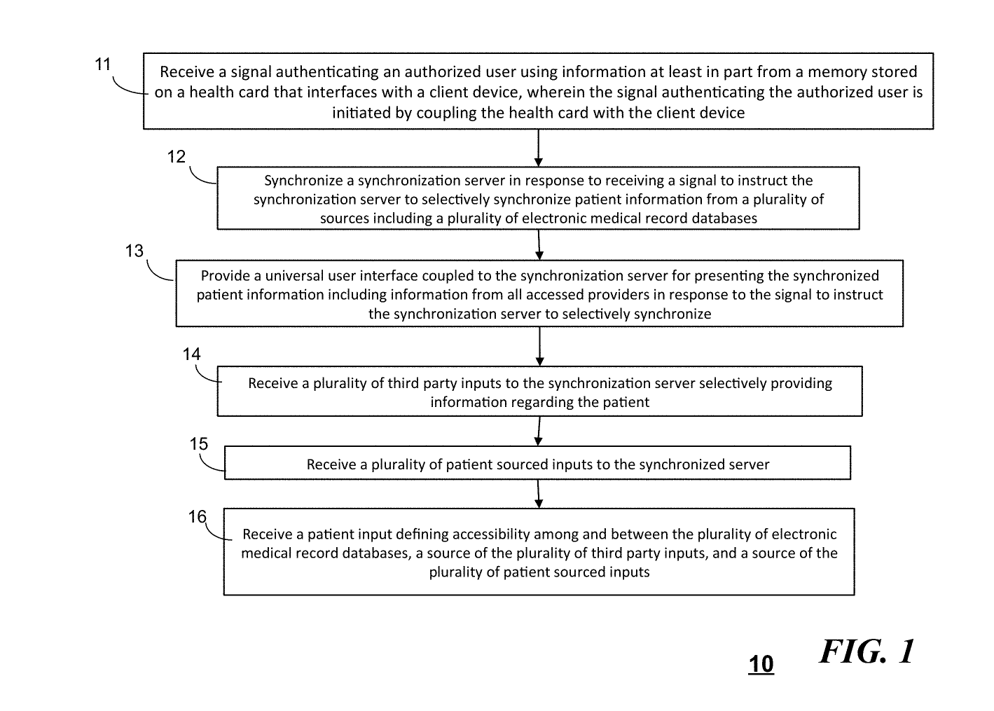

Referring to FIG. 1, a flow chart illustrating a method 10 in accordance with some embodiments can include the steps of receiving a signal authenticating an authorized user using information at least in part from a memory stored on a health card that interfaces with a client device at step 11, wherein the signal authenticating the authorized user is initiated by coupling the health card with the client device and synchronizing at step 12 a synchronization server in response to receiving a signal to instruct the synchronization server to selectively synchronize patient information from a plurality of sources including a plurality of electronic medical record databases. The method 10 can further include the step 13 of providing a universal user interface coupled to the synchronization server for presenting the synchronized patient information including information from all accessed providers in response to the signal to instruct the synchronization server to selectively synchronize. Optionally, the method 10 can further include the step 14 of receiving a plurality of third party inputs to the synchronization server selectively providing information regarding the patient, the step 15 of receiving a plurality of patient sourced inputs to the synchronized server, and the step 16 of receiving a patient input defining accessibility among and between the plurality of electronic medical record databases, a source of the plurality of third party inputs, and a source of the plurality of patient sourced inputs.

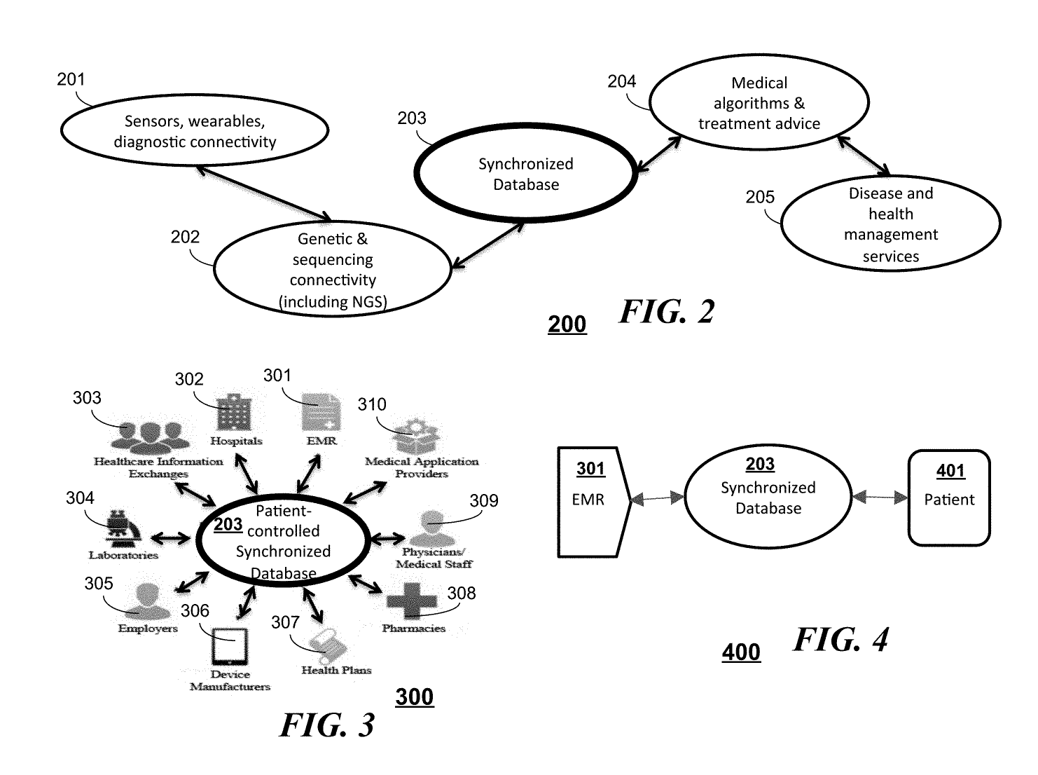

Referring to FIG. 2, a system 200 in accordance with some embodiments can include an integrated information system including a synchronized database 203 that is communicatively coupled to sensors or wearable or other diagnostic devices 201 as well as optionally coupled to genetic or sequencing information sources 202 such as NGS or next generation sequencing. The synchronized server can also be coupled to devices 204 that provide medical algorithms or treatment advice from healthcare providers or from artificial intelligence sources and from other devices 205 that provide disease and health management services.

In a more generic sense, such a system would be described as a patient-centric universal health recording and payment system having a synchronized server with synchronized patient information accessible and controllable by a patient, a plurality of electronic medical record databases selectively synchronized with the synchronized server, and a universal user interface coupled to the synchronized server for presenting the synchronized patient information including information from all accessed providers. The system can further include a plurality of third party inputs to the synchronized server selectively providing information regarding the patient with respect to one or more, for example, among telemedicine information, nutrition intake information, or genetics information. The system can also include a plurality of patient sourced inputs to the synchronized server that selectively provides information such as one or more among patient monitored data, patient record outcome measures, or caretaker record outcome measures. As this is a patient-centric system, the system can further include a patient input defining accessibility among and between the plurality of electronic medical record databases, a source of the plurality of third party inputs, and a source of the plurality of patient sourced inputs. In some embodiments, the system can include a patient health card providing patient authentication or patient identification as a condition precedent to enabling the patient input defining accessibility. In some embodiments, the system can include a patient health card providing patient authentication or patient identification as a condition precedent to enabling the patient input defining accessibility and where the patient health card further provides funding for payments of health care services or other services. In some embodiments, the patient health card provides funding for payments using cryptocurrency. In some embodiments payment transactions are initiated using cryptocurrency using the patient health card coupled to a client device where the transaction is stored using a direct acyclic graph.

In some embodiment the plurality of third party inputs can include one or more among third party telemedicine, third party medical algorithms, third party medical treatment advice, third party disease and health management services, third party provided genetic sequencing information, and third party nutritional intake information. In some embodiments, the plurality of patient sourced inputs can include one or more among data obtained from passive remote data collection from the patient, remote diagnostics from the patient, wearables from the patient, fitness trackers from the patient, self recorded inputs from the patient, and caretaker inputs observed about the patient. In some embodiments, the plurality of electronic medical record (EMR) databases can include one or more among a primary care physician EMR, a hospital EMR, a specialist EMR and a skilled nursing facility EMR. In some embodiments, the system can further include an Electronic Data Capture input to the universal user interface or the synchronized server enabling access to patient information for clinical research.

In some embodiments, the health card enables transactions and transfers of value among a patient account and a service provider and yet other embodiments the health card enables transactions and transfers of value among a patient account, a service provider, and an insurance provider.

Referring to FIG. 3, a system 300 in accordance with some embodiments can include the patient controlled synchronized database 203 coupled to a myriad of device and information sources for an individual patient such as an EMR 301, a hospital 302, a healthcare information exchange 303, a laboratory 304, an employer 305, a device manufacturer 306, a health care plan 307, a pharmacy 308, a physician or health care provider 309, or a medical application provider 310.

Referring to FIG. 4, a system 400 in accordance with some embodiments can include at a high level, the patient controlled synchronized database 203 communicatively coupled and controlled and managed to a certain extent by a patient 401 on one end and a Electronic Medical Record or EMR 301 on another end. A single EMR or medical record is not typically a current practical real world result as numerous providers have their own electronic medical records for each patient. Operationally, the patient 401 can log into their patient portal through the synchronized database 203. The data in the synchronized database 203 is synchronized from the EMR 301 to the synchronized database 203 and the patient 401 has the ability to share the data with whomever they choose.

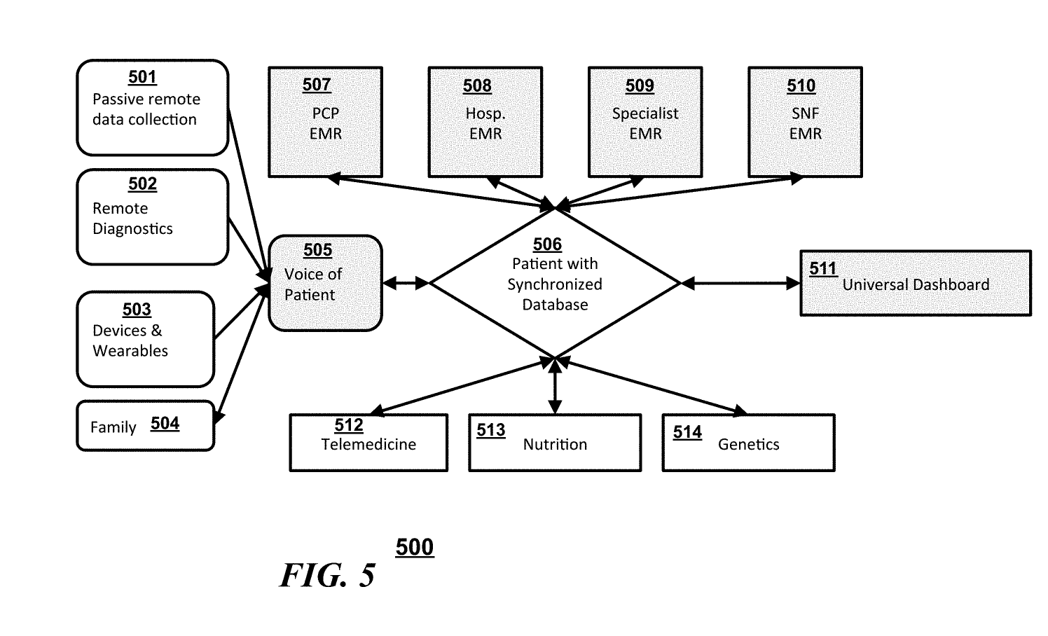

Referring to FIG. 5, a system 500 in accordance with some embodiments can include a synchronized database 506 which communicates with a “voice of a patient” 505 (which can be a plurality of patient sourced inputs), a number EMRs which can be disparate EMR databases, a client device having a universal dashboard 511, and a plurality of third party inputs. The “voice of the patient” 505 can simply pass through information obtained from or about the patient via passive remote data collection 501, from remote diagnostics 502, from devices or wearables 503, or from family members or care providers 504. In some embodiments, the “voice of the patient” 505 can integrate some or all of the patient sourced information and provide a normalized data set to the synchronized database 506. The different EMRs that can be synchronized with the synchronized database 506 can include, but is not limited to a primary care provider (PCP) EMR 507, a hospital EMR 508, a specialist EMR 509, and/or a skilled nursing facility (SNF) EMR 510. The third party inputs to the synchronized server 506 can include sources from telemedicine providers 512, nutritionists 513, and/or third party genetic analysts or counselors 514. Operationally, the patient can control the flow and integration of information into the synchronized server and to other provider information databases through the universal dashboard 511. In an oncology patient type setting, the patient or authorized care provider can control and coordinate the care and information coordination via the universal dashboard 511 and the synchronized database 506 to obtain the information and holistic information from the voice of the patient 505 and from the different EMRs (507, 508, 509, and 510).

Ensuring continuity of care is also a function of the care coordination focus. A patient centered medical home (PCMH) has the responsibility to assist patients with their care beyond the clinical walls and follow-up appropriately. Tracking and testing, especially for abnormal labs and imaging studies, and physician referrals is paramount for ensuring successful continuity of care. Managing transitions from and between specialty clinics or physicians, acute settings, post-acute settings, and community organizations is critical for effective care and meeting the objectives of the PCMH. The PCMH emphasizes that all clinicians work as a team at the top of their licenses. The physician will function as a part of a team with the focus on communication, collaboration, and care coordination instead of the segmented and siloed practice of today. Mid-level providers may see low-risk patients, with a focus on prevention and healthy lifestyle practices. This will contribute to additional office overhead but will enhance efficiency, quality, and satisfaction. Shared savings or health care system subsidies for the participating PCP may offset any additional costs.

The PCMH is also a practice that focuses on clinical and operational improvement quantitatively. The team uses current data to identify areas of opportunity to improve care and prevention. In addition to identifying vulnerable populations to measure quality performance, all patients are checked to ensure routine screenings and other preventive services. Chronic disease performance metrics also help the practice determine its effectiveness to improve outcomes. Practices also measure areas of utilization to determine efficiency and lower costs. Measuring and improving communication and patient experience are priorities to ensure patients understand their care. Measuring and understanding patient engagement is critical. Not only do these practices set improvement targets, but high performing PCMHs can easily demonstrate continuous improvement over time, exhibiting high clinical quality and safety using the systems and methods described herein. Robust and specific documentation, as well as the latest EHR, adhering to the most current meaningful use standards, is essential to achieve these goals and the ability to measure them. The embodiments herein can facilitate applying the most up to date standards of care via clinical decision support and patient decision support and shared decision making when all the appropriate data is available for decisions including treatment options with side effects lists and costs.

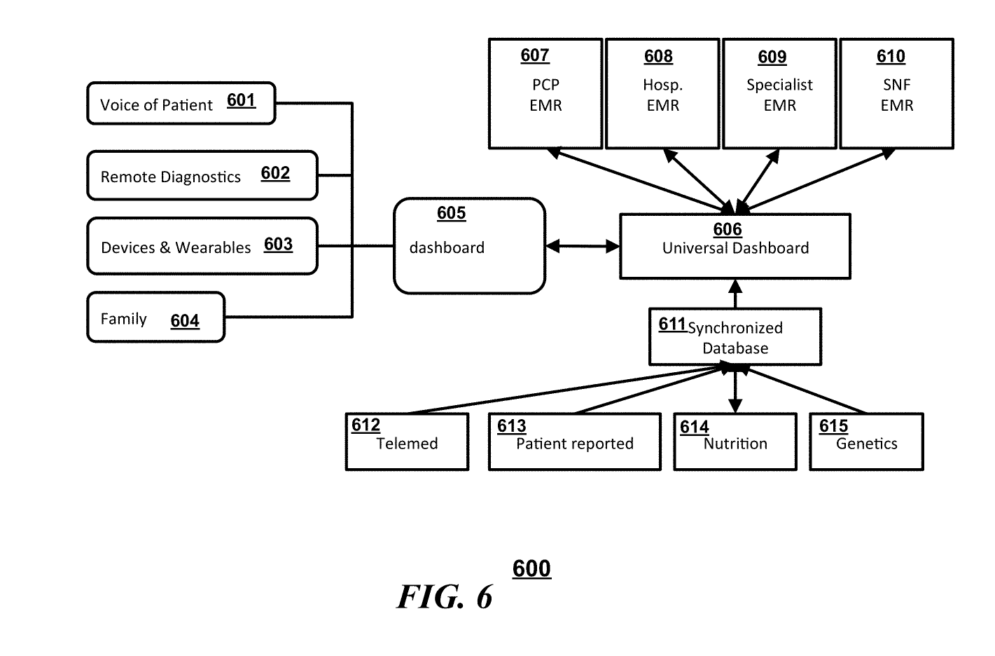

Referring to FIG. 6, a system 600 similar to system 500 of FIG. 5 in accordance with some embodiments can include a synchronized database 611, which communicates with a universal dashboard 606 (which can be part of a client device). The dashboard 606 can be communicatively coupled to a plurality of patient sourced inputs) via another dashboard 605. The universal dashboard 606 can be communicatively coupled to a number EMRs which can be disparate EMR databases. In some embodiments, the “voice of the patient” 505 can integrate some or all of the patient sourced information and provide a normalized data set to the synchronized database 506. The different EMRs that can be synchronized with the synchronized database 611 via the dashboard 606 can include, but is not limited to a primary care provider (PCP) EMR 607, a hospital EMR 608, a specialist EMR 609, and/or a skilled nursing facility (SNF) EMR 610. Third party inputs to the synchronized server 611 can include sources from telemedicine providers 612, patient reported information 613, nutritionists 614, and/or third party genetic analysts or counselors 615. Patient sourced information can also be integrated, interface, and reported as inputs to the synchronized database 611 via the dashboards 606 and 605 and can include “voice of the patient” 601, remote diagnostics 602, information from devices or wearables 603, or information from family members or care providers 604. Operationally, the patient can control the flow and integration of information into the synchronized server and to other provider information databases through the universal dashboard 606 and/or the dashboard 605.

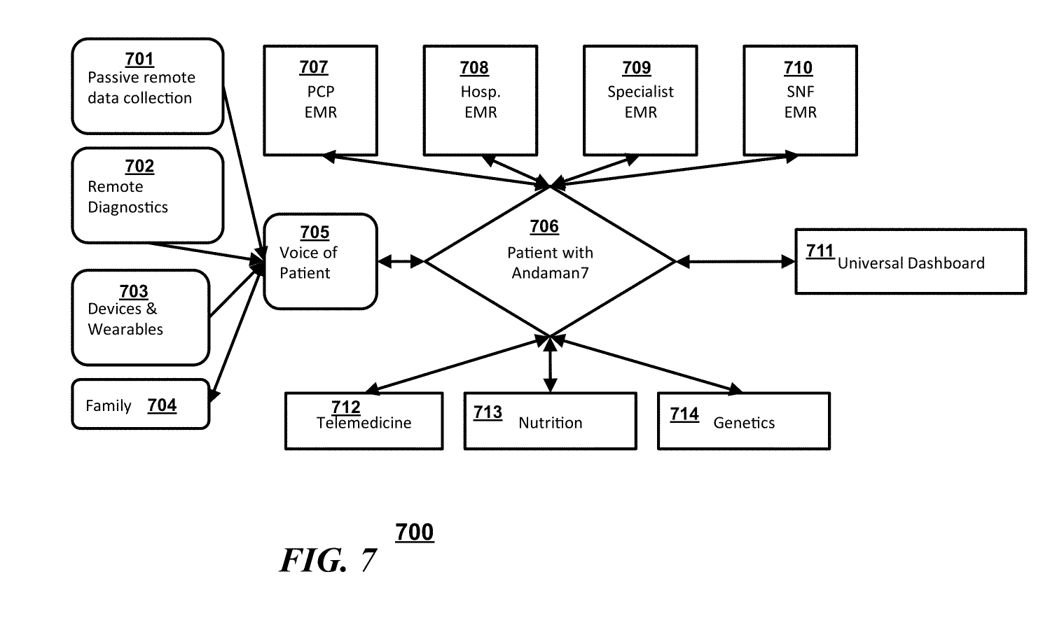

Referring to FIG. 7, a system 700 similar to system 500 of FIG. 5 and in accordance with some embodiments can include a synchronized database 706 which communicates with a “voice of a patient” 705 (which can be a plurality of patient sourced inputs), a number EMRs which can be disparate EMR databases, a client device having a universal dashboard 711, and a plurality of third party inputs. The “voice of the patient” 705 can simply pass through information obtained from or about the patient via passive remote data collection 701, from remote diagnostics 702, from devices or wearables 703, or from family members or care providers 704. In some embodiments, the “voice of the patient” 705 can integrate some or all of the patient sourced information and provide a normalized data set to the synchronized database 706. The different EMRs that can be synchronized with the synchronized database 706 can include, but is not limited to a primary care provider (PCP) EMR 707, a hospital EMR 708, a specialist EMR 709, and/or a skilled nursing facility (SNF) EMR 710. The third party inputs to the synchronized server 706 can include sources from telemedicine providers 712, nutritionists 713, and/or third party genetic analysts or counselors 714. Operationally, the patient can control the flow and integration of information into the synchronized server and to other provider information databases through the universal dashboard 711.

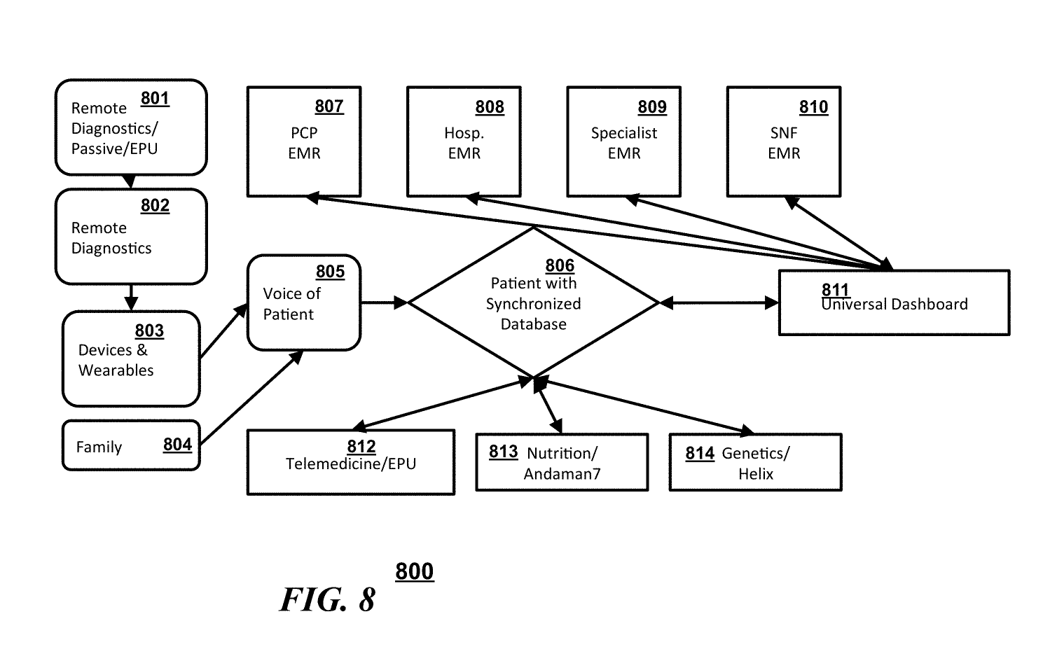

Referring to FIG. 8, a system 800 in accordance with some embodiments can include a synchronized database 806 which communicates with a “voice of a patient” 805 (which can be a plurality of patient sourced inputs), a number EMRs which can be disparate EMR databases, a client device having a universal dashboard 811, and a plurality of third party inputs. The “voice of the patient” 805 can simply pass through information obtained from or about the patient from devices or wearables 803, or from family members or care providers 804. Information from passive remote data collection 801 or from remote diagnostics 802 can also be uploaded to the synchronized database 806 via the voice of the patient 805 interface. In some embodiments, the “voice of the patient” 505 can integrate some or all of the patient sourced information and provide a normalized data set to the synchronized database 806. The different EMRs that can be synchronized with the synchronized database 806 via the universal dashboard 811. The different EMRs can include, but is not limited to a primary care provider (PCP) EMR 807, a hospital EMR 808, a specialist EMR 809, and/or a skilled nursing facility (SNF) EMR 810. The third party inputs to the synchronized server 806 can include sources from telemedicine providers 812, nutritionists 813, and/or third party genetic analysts or counselors 814. Operationally, the patient can control the flow and integration of information into the synchronized server and to other provider information databases through the universal dashboard 811.

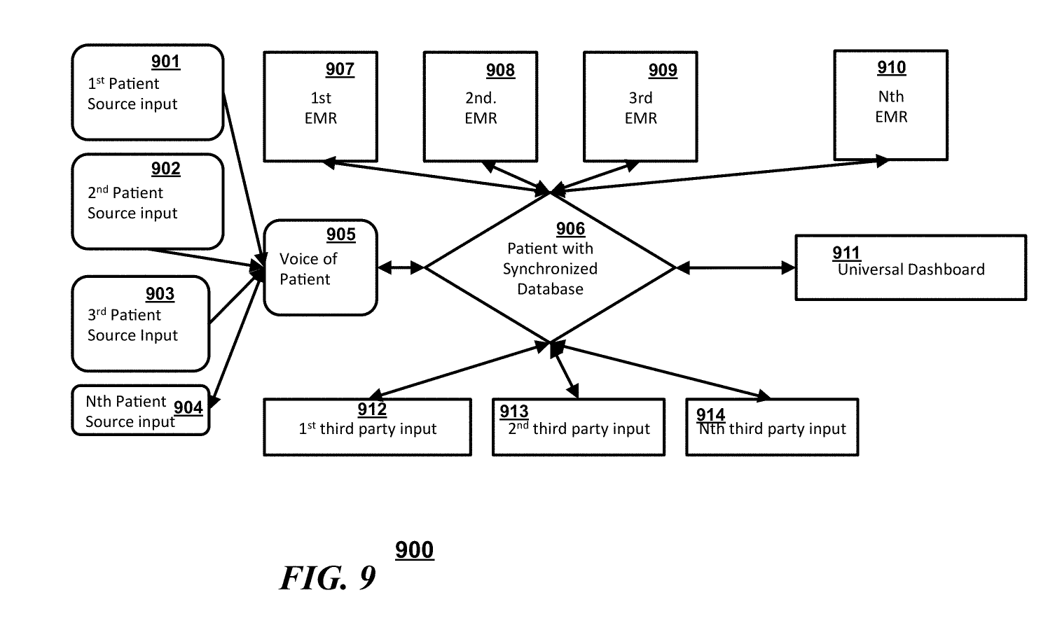

Referring to FIG. 9, a system 900 in accordance with some embodiments can include a synchronized database 906 which communicates with a “voice of a patient” 905 (which can be a plurality of patient sourced inputs), a number EMRs which can be disparate EMR databases, a client device having a universal dashboard 911, and a plurality of third party inputs. The “voice of the patient” 905 can simply pass through information obtained from or about the patient from from any number of first through nth patient sourced inputs (901, 902, 903, or 904). In some embodiments, the “voice of the patient” 905 can integrate some or all of the patient sourced information and provide a normalized data set to the synchronized database 906. The different EMRs that can be synchronized with the synchronized database 906 where any number of the different EMRs can include, but is not limited first through nth EMRs (907, 908, 909, and/or 910). The third party inputs to the synchronized server 906 can include sources from first through nth third party sources (912, 913, and/or 914). Operationally, the patient can control the flow and integration of information into the synchronized server and to other provider information databases through the universal dashboard 911.

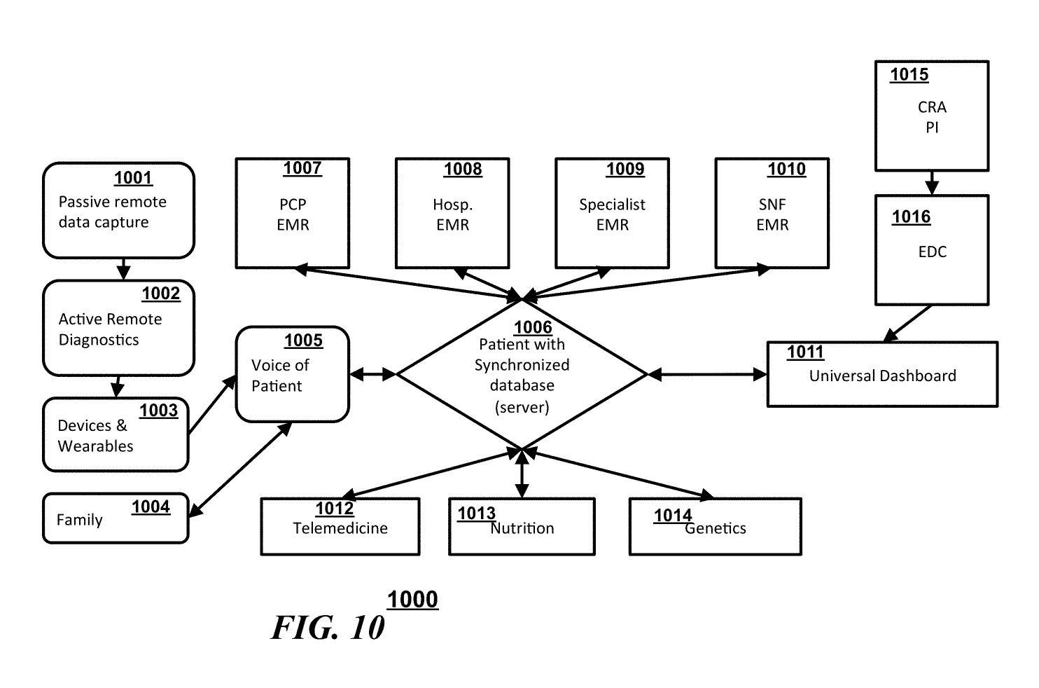

Referring to FIG. 10, a system 1000 similar to system 500 of FIG. 5 and in accordance with some embodiments can include a synchronized database 1006 which communicates with a “voice of a patient” 1005 (which can be a plurality of patient sourced inputs), a number EMRs which can be disparate EMR databases, a client device having a universal dashboard 1011, and a plurality of third party inputs. The “voice of the patient” 1005 can simply pass through information obtained from or about the patient via passive remote data collection 1001, from remote diagnostics 1002, from devices or wearables 1003, or from family members or care providers 1004. In some embodiments, the “voice of the patient” 1005 can integrate some or all of the patient sourced information and provide a normalized data set to the synchronized database 1006. The different EMRs that can be synchronized with the synchronized database 1006 can include, but is not limited to a primary care provider (PCP) EMR 1007, a hospital EMR 1008, a specialist EMR 1009, and/or a skilled nursing facility (SNF) EMR 1010. The third party inputs to the synchronized server 1006 can include sources from telemedicine providers 1012, nutritionists 1013, and/or third party genetic analysts or counselors 1014. In some embodiments, the system can further include an interface to research facilities via the universal dashboard 1011. For example, a clinical research associate (CRA) or a principal investigator (PI) 1015 doing clinical research can provide or extract information via the universal dashboard 1011 from or to the synchronized database 1006. Similarly, an electronic data capture system 1016 can provide or extract information via the universal dashboard 1011 from or to the synchronized database 1006.

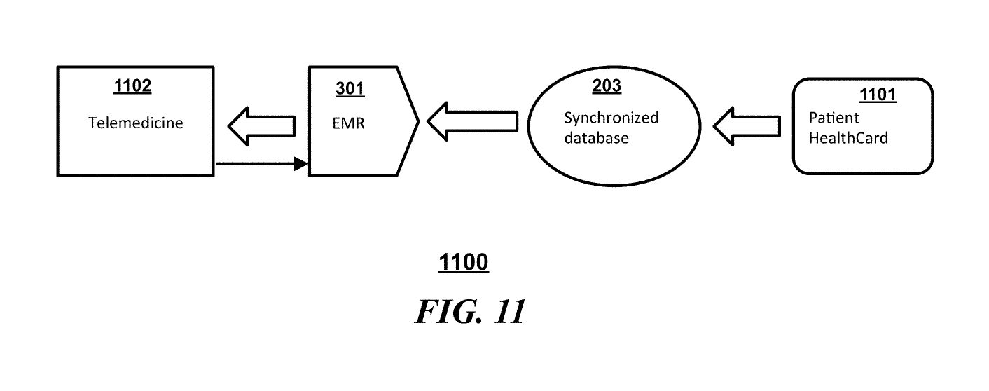

Referring to FIG. 11, a system 1100 in accordance with some embodiments can include at a high level, the patient controlled synchronized database 203 communicatively coupled and controlled and managed to a certain extent by a patient electronic card 1101 on one end and a Electronic Medical Record or EMR 301 on another end. A single EMR or medical record is not typically a current practical real world result as numerous providers have their own electronic medical records for each patient. For example, one EMR can be from a telemedicine provider 1102. Operationally, the patient electronic card 1101 can be used to log a patient into their patient portal through the synchronized database 203. The data in the synchronized database 203 is synchronized from the EMR 301 to the synchronized database 203. Via the patient electronic card 1101 and the synchronized database 203, the EMR 301 can be authorized to allow information to be forward and received from a third party such as the telemedicine provider (or to share the data with whomever the patient chooses).

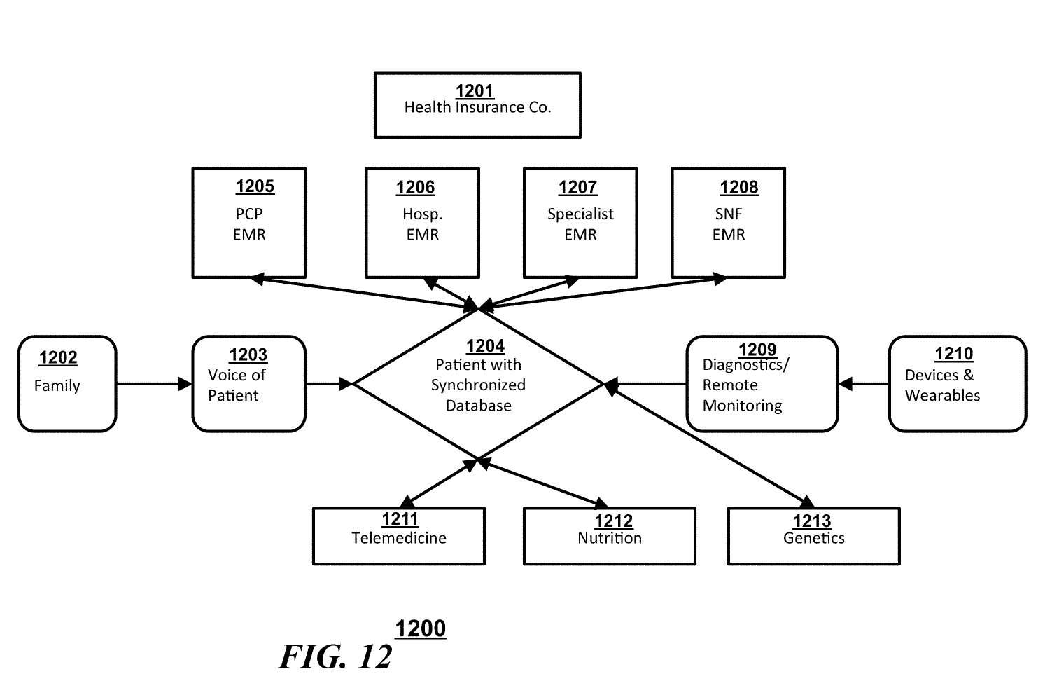

Referring to FIG. 12, a system 1200 in accordance with some embodiments can include a synchronized database 1204 which communicates with a “voice of a patient” 1203 (which can be a plurality of patient sourced inputs), a number EMRs which can be disparate EMR databases, and a plurality of third party inputs. The “voice of the patient” 1203 can simply pass through information obtained from or about the patient from family members or care providers 1202. Other patient sourced inputs can include remote diagnostics or monitoring 1209 or information from devices or wearables 1210. The different EMRs that can be synchronized with the synchronized database 1204 can include, but is not limited to a primary care provider (PCP) EMR 1205, a hospital EMR 1206, a specialist EMR 1207, and/or a skilled nursing facility (SNF) EMR 1208. The third party inputs to the synchronized server 1204 can include sources from telemedicine providers 1211, nutritionists 1212, and/or third party genetic analysts or counselors 1213. Operationally, the patient can control the flow and integration of information into the synchronized server and to other provider information databases through the universal dashboard (not shown) to allow a health insurance company 1201 to monitor the healthcare process in a holistic fashion.

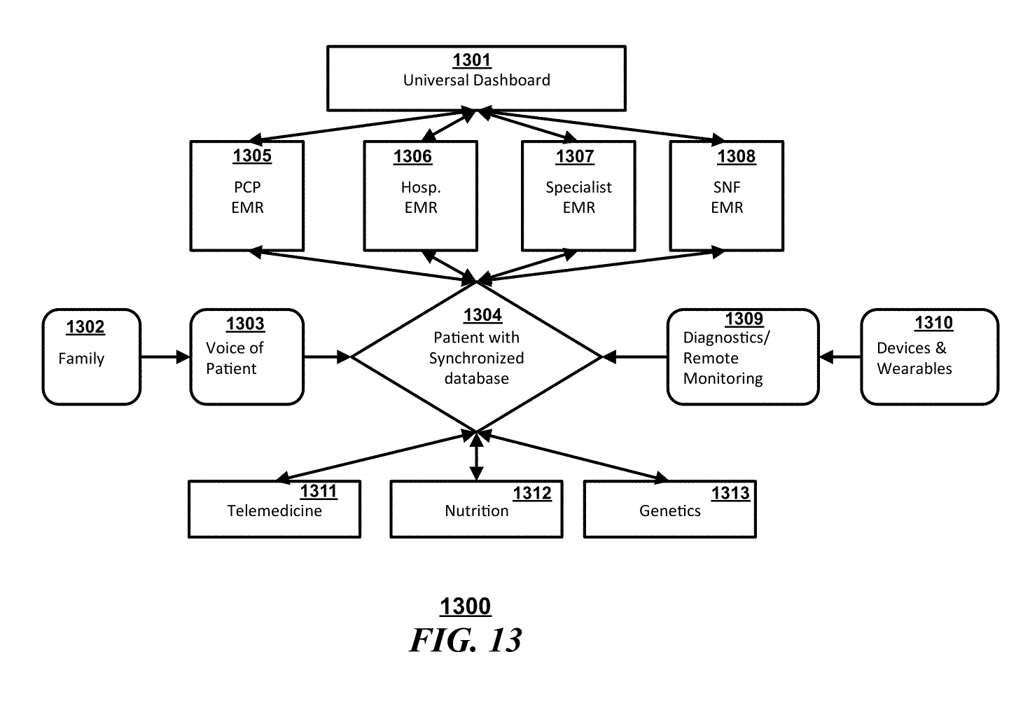

Referring to FIG. 13, a system 1300 in accordance with some embodiments can include a synchronized database 1304 which communicates with a “voice of a patient” 1303 (which can be a plurality of patient sourced inputs), a number EMRs which can be disparate EMR databases, and a plurality of third party inputs. The “voice of the patient” 1303 can simply pass through information obtained from or about the patient from family members or care providers 1302. Other patient sourced inputs can include remote diagnostics or monitoring 1309 or information from devices or wearables 1310. The different EMRs that can be synchronized with the synchronized database 1304 can include, but is not limited to a primary care provider (PCP) EMR 1305, a hospital EMR 1306, a specialist EMR 1307, and/or a skilled nursing facility (SNF) EMR 1308. The third party inputs to the synchronized server 1304 can include sources from telemedicine providers 1311, nutritionists 1312, and/or third party genetic analysts or counselors 1213. Operationally, the patient, a health care provider or a health care company or insurance provider can control the flow and integration of information into the synchronized server and to other provider information databases through the universal dashboard 1301 (coupled to the EMRs) to enable the integrated monitoring of the healthcare process in a holistic fashion.

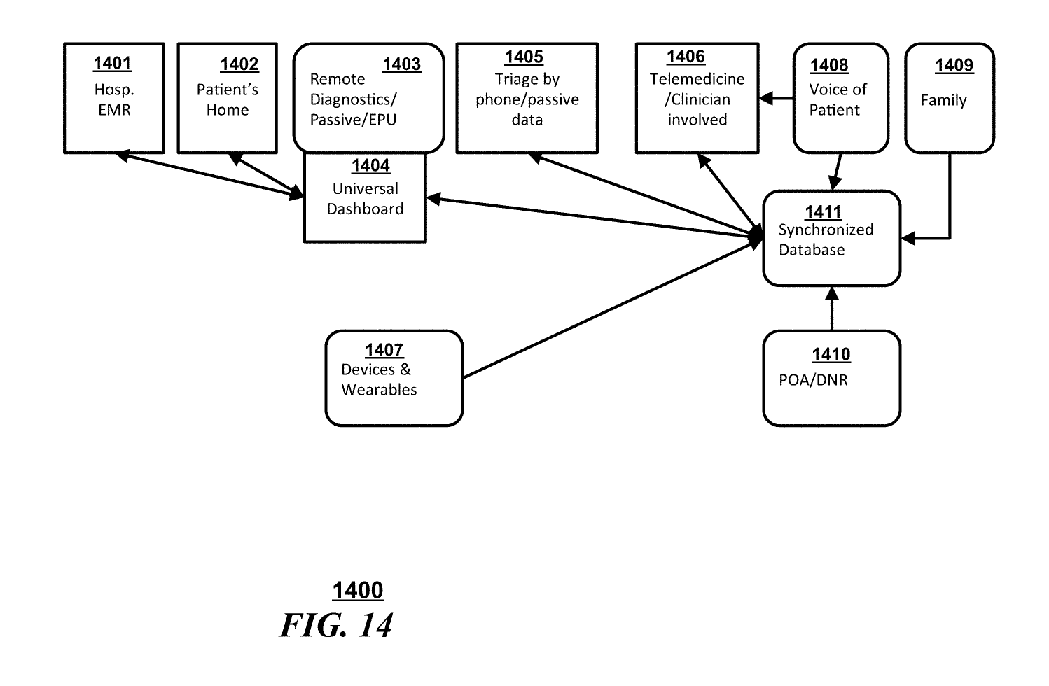

Referring to FIG. 14, a system 1400 can include a synchronized database or server 1411 that receives various inputs and coordinates transfer of patient information to or from other entities or sources based on permissions stored at the synchronized database 1411 and otherwise based on patient instructions. For example, a Power of Attorney (POA) or Do Not Resuscitate (DNR) order 1410 can be stored and updated at the synchronized database 1411. As in other examples, inputs from family members (or other designated care takers) 1409 provide inputs to the database 1411. A “voice of the patient” 1408 can also serve as an subjective input from the patient for other care providers to refer to as part of their overall or holistic diagnosis or review. Other inputs can include devices or wearables 1407 that monitor the patient continuously or periodically or intermittently as desired. Third party care providers or sources can utilize the sychnronized database to either provide additional inputs or to analyze the existing information on the synchronized database 1411. Such third party care provider or sources can access the synchronized database 1411 either directly or via a universal dashboard 1404. In this embodiment, a telemedicine or clinician 1406 or a remote source 1405 (such as an 911 emergency responder or emergency room doctor or nurse can provide triage by phone to the synchronized database 1411 directly while hospital EMR 1401 or patient's home 1402 or a remote diagnostic (or early pregnancy unit (EPU)) 1402 can provide inputs and outputs to to the synchronized server 1411 via the universal dashboard 1404.

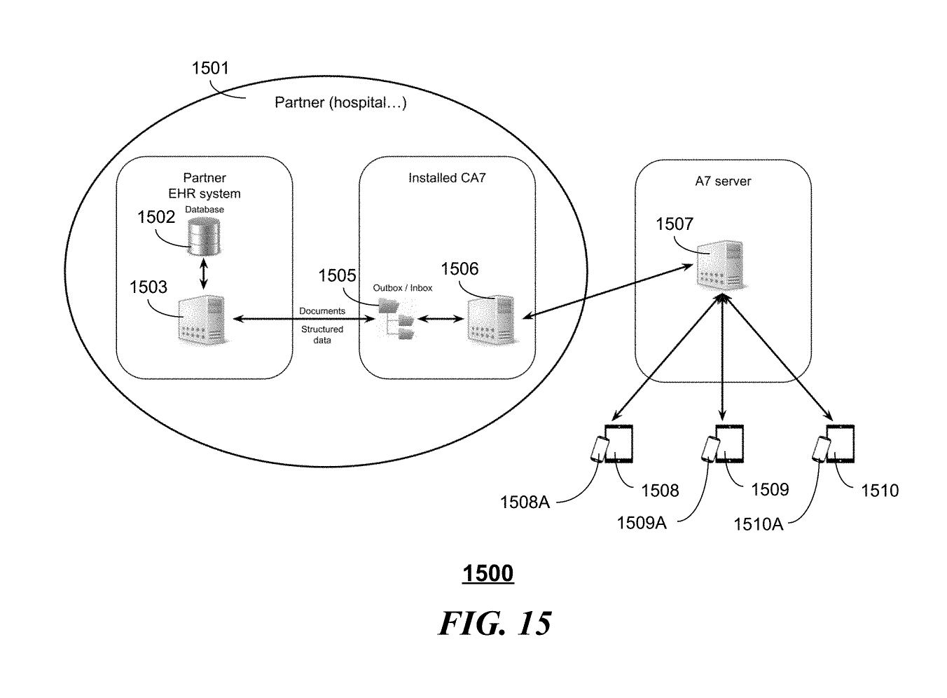

Referring to FIG. 15, a block diagram of a system 1500 for providing patient-centric universal health recording and payment can include a server 1507 having a computer program or programs enabling the authorized and secure access from one or more client devices 1508, 1509, or 1510 and from third party sources such as a hospital 1501. The respective client devices utilize respective health cards 1508A, 1509A, or 1510A to obtain secure and authenticated access to the server 1507 and third party sources. The hospital 1501 can include a electronic medical record database 1502 and server 1502 that communicates with an application or file structure 1505 on the same server 1502 or optionally on a separate server 1506 that is configured to securely communicate in a synchronized fashion with the server 1507 in accordance with the embodiments herein. The computer instructions on the servers 1503 and/or 1506 can enable the integration of a hospital or other party's EMR data warehouse with synchronized server 1507 and authorized client devices. Such a system would likely include card access software installed at the hospital EMR server or servers 1506 and/or 1503 that enables secure and synchronized communication with the synchronized server 1507. In some embodiments, the synchronization between servers can be fully automated. In other embodiments, the synchronization can be done when a patient asks authorized hospital staff to selectively activate synchronization between the hospital EMR 1502 and the server 1507. The patient, through the authorized and authenticated client device 1508 using their health card 1508A, can then define how the synchronized data is shared with third parties as desired.

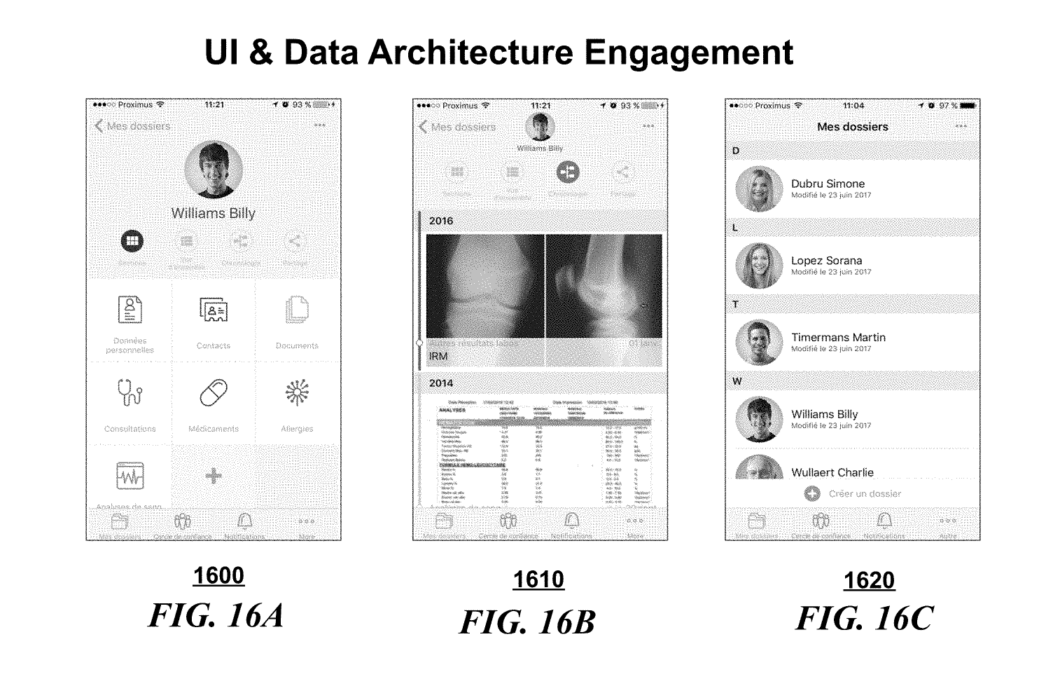

Referring to FIGS. 16A, 16B, and 16C, various respective user interfaces 1600, 1610 and 1620 on client devices used by either a patient or a health care provider or clinician illustrate how they might appear on such devices. The user interface 1600 of FIG. 16A illustrates a customizable or tailored home page that includes one or more icons on the home page to focus on quick access on information useful for a particular a patient including contacts, documents, consultations, medications, allergies, consultations or appointments. The user interface 1610 of FIG. 16B can include files that can include images such as x-rays, CT-scans, MRIs, and blood work analysis for example. The user interface 1620 of FIG. 16C can illustrate a ‘circle of trust’ listing that includes individuals who the patient wishes to share their data with (or vice-versa). In other words, the listing of the circle of trust can include one or more among physicians, insurers, care providers, family, and friends which the patient can selectively grant access to all or a portion of the patient's medical information.

It is no longer an option for physicians to ignore the patient experience. Payers, including the federal government (through the Centers for Medicare & Medicaid Services or CMS) have made it clear that only those providers with acceptable levels of patient satisfaction will be allowed to receive rewards and/or participate in future reimbursement programs. While not widely adopted beyond large physician groups and healthcare aligned organizations, physician experience/patient satisfaction is a critical element of any compensation methodology. As physicians aggregate into larger and more sophisticated groups, standardized methods of patient satisfaction reporting are necessary and a focus on high benchmarks and continuous improvement is a key component. The embodiments herein further enable the recording of such patient satisfaction levels to enable payers such as health insurance companies or the government to appropriately reward (or punish) health care providers accordingly.

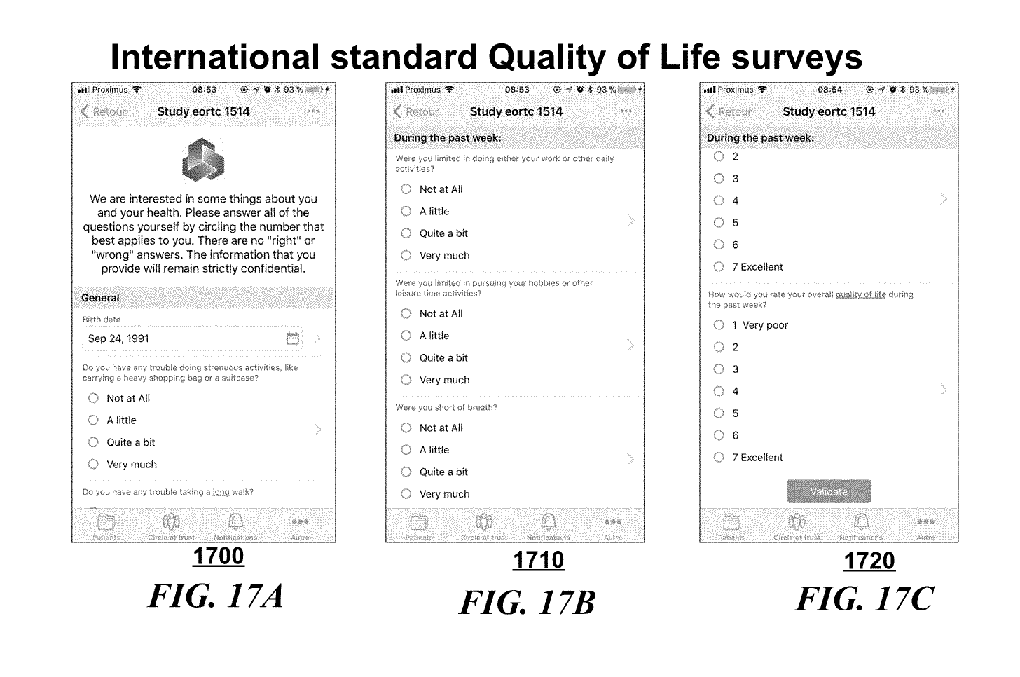

Referring to FIGS. 17A, 17B, and 17C illustrate respective portions 1700, 1710, and 1730 of a user interface showing a quality of life survey for a patient. Such surveys having subjective and objective data can be used with other information to analyze the state of well being for a particular patient or for classes of individuals in clinical studies. The amount of healthcare data that will be transparently available going forward is voluminous. Collaborators, associations, and the Federal government are all working toward the common goal of interoperable, aggregated, and uniform data sets to study and improve population health.

Regardless if work will be completed through several groups, or through one centralized agency, care will be delivered based on analytical and qualitative data with cost to cure considerations. The end result will be evidence-based strategies to maintain wellness, encourage prevention of disease in at-risk populations, and direct treatments based on outcomes and costs. The Office of the National Coordinator for Health Information Technology (ONC) has illustrated the wealth of information and resulting knowledge.

The primary meaning of consumerism in healthcare is a movement in which patients are involved in their own care decisions through a partnership with physicians. Consumerism encourages health information empowerment and a basic understanding of body functions, chronic disease, and disease prevention. It involves patient education, through clinicians, to increase patients' involvement in the decision-making process.

Patient involvement and engagement in care decisions has dramatically increased in the last decade. This has led to the development and use of health and wellness apps, formation of disease-specific websites, and patient-promoted research. The various embodiments disclosed herein will only further promote these states goals and trends.

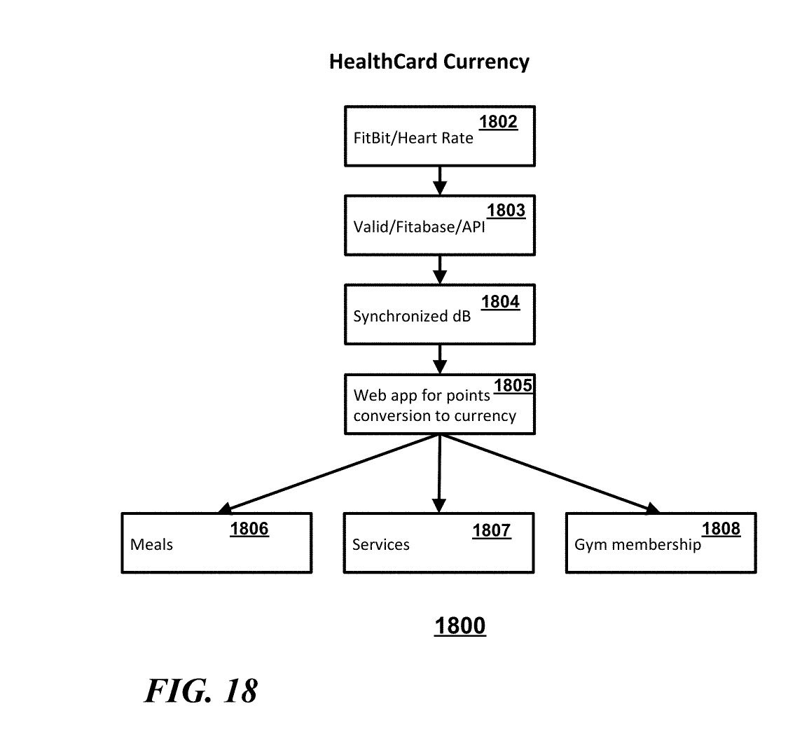

One of the goals in using the patient-centric universal health recording and payment system is to achieve better, faster and lower cost treatments by collecting real world evidence (RWE) about the treatments being used. RWE is Evidence collected outside of the clinical setting, which is where most people spend most of their time. Referring to FIG. 18, a method 1800 of collecting such RWE can include devices and incentives that encourage patients or users to authorize and submit their information for points and subsequent conversion to currency that can be used with vendors in such an ecosystem such as meal providers 1806, service providers 1807 or gym membership providers 1808. For example, a patient can track and submit their heart rate data 1802 or other monitored data using a FitBit' or other suitable monitoring device via an application program interface 1803 to a synchronized database 1804. A system can be implemented that assigns points for just the mere submission of data or even provides bonus points for healthful improvements indicated by the data over time. Such points can be converted at step 1805 and subsequently utilized by the patient at the different providers 1806, 1807, and 1808.

Similar schemes can be used to track the life cycle of a medication or treatment. During development a company can easily spend $1.5B and 7 years on average to create a medicine. Once the medicine is approved and launched, it can turn into a $5B revenue stream each year for such a pharmaceutical company. Payers and doctors are realizing that these treatments don't necessarily work for everyone and want more data for more personalized and targeted use.

So payers and regulators are requiring medicine vendors to collect more RWE today than in the past. Use of the synchronized database as described herein can help fill the gap once a product is launched in the market. In other words, the embodiments herein can be focused on ‘phase 4’. Phase 4 is more patient centered, where RWE and Patient Record Outcome Measures or PROM is more valued.

However, the greatest impact can be realized by identifying and targeting the broader population who are currently healthy, but at-risk for future diseases. Data collection and analyses are essential to identify patients at risk. One difficulty, mentioned by several CMS Medicare Shared Savings Program (MSSP) and Accountable Care Organization (ACO) Pilot participants, is the significant lag in Medicare and Medicaid claims prohibiting the monitoring of readmissions and the at-risk population.

The increase in patient involvement in their care decisions, hence consumerism, also positively impacts overall satisfaction. Patients prefer to have control over their health decisions. According to a HealthLeaders Media Population Health Survey, two-thirds (66%) of healthcare organizations expect to invest in patient engagement programs, improving access by encouraging patients to become more aware of their own health status and their role in maintaining good health. Once again, the embodiments herein can be configured, designed and customized with these stated goals in mind.

Various embodiments of the present disclosure can be implemented on an information processing system. The information processing system is capable of implementing and/or performing any of the functionality set forth above. Any suitably configured processing system can be used as the information processing system in embodiments of the present disclosure. The information processing system is operational with numerous other general purpose or special purpose computing system environments, networks, or configurations. Examples of well-known computing systems, environments, and/or configurations that may be suitable for use with the information processing system include, but are not limited to, personal computer systems, server computer systems, thin clients, hand-held or laptop devices, multiprocessor systems, mobile devices, microprocessor-based systems, set top boxes, programmable consumer electronics, network PCs, minicomputer systems, mainframe computer systems, Internet-enabled television, and distributed cloud computing environments that include any of the above systems or devices, and the like.

For example, a user with a mobile device may be in communication with a server configured to implement the health recording and payment system, according to an embodiment of the present disclosure. The mobile device can be, for example, a multi-modal wireless communication device, such as a “smart” phone, configured to store and execute mobile device applications (“apps”) or a laptop or notepad device configured to store and execute similar applications. Such a wireless communication device communicates with a wireless voice or data network using suitable wireless communications protocols. The user signs in and access the secure synchronized database service layer, including the various modules described above. The service layer in turn communicates with various databases, such as a user level DB, a generic content repository, and a data repository. The generic content repository may, for example, contain enterprise documents, internal data repositories, personal data repositories, and 3^{rd} party data repositories. The service layer queries these databases and presents responses back to the user based upon the rules and interactions of the various modules.

The system which can be part of a patient-centric universal health recording and payment system may include, inter alia, various hardware components such as processing circuitry executing modules that may be described in the general context of computer system-executable instructions, such as program modules, being executed by the system. Generally, program modules can include routines, programs, objects, components, logic, data structures, and so on that perform particular tasks or implement particular abstract data types. The modules may be practiced in various computing environments such as conventional and distributed cloud computing environments where tasks are performed by remote processing devices that are linked through a communications network. In a distributed cloud computing environment, program modules may be located in both local and remote computer system storage media including memory storage devices. Program modules generally carry out the functions and/or methodologies of embodiments of the present disclosure, as described above.

In some embodiments, a system includes at least one memory and at least one processor of a computer system communicatively coupled to the at least one memory. The at least one processor can be configured to perform a method including methods described above.

According yet to another embodiment of the present disclosure, a computer readable storage medium comprises computer instructions which, responsive to being executed by one or more processors, cause the one or more processors to perform operations as described in the methods or systems above or elsewhere herein.

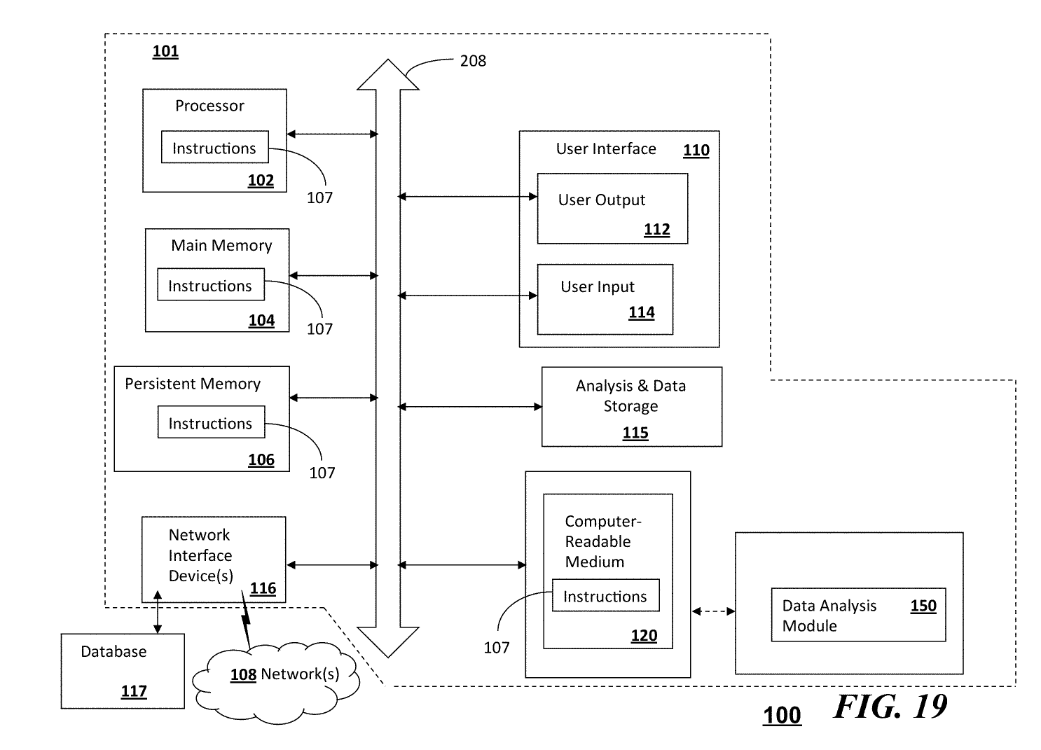

As shown in FIG. 19, an information processing system 101 of a system 100 can be communicatively coupled with the data analysis module 150 and a group of client or other devices, or coupled to a presentation device for display at any location at a terminal or server location. According to this example, at least one processor 102, responsive to executing instructions 107, performs operations to communicate with the data analysis module 150 via a bus architecture 208, as shown. The at least one processor 102 is communicatively coupled with main memory 104, persistent memory 106, and a computer readable medium 120. The processor 102 is communicatively coupled with an Analysis & Data Storage 115 that, according to various implementations, can maintain stored information used by, for example, the data analysis module 150 and more generally used by the information processing system 100. Optionally, this stored information can be received from the client or other devices. For example, this stored information can be received periodically from the client devices and updated or processed over time in the Analysis & Data Storage 115. Additionally, according to another example, a history log can be maintained or stored in the Analysis & Data Storage 115 of the information processed over time. The data analysis module 150, and the information processing system 100, can use the information from the history log such as in the analysis process and in making decisions related to determining whether data measured is considered an outlier or not for any number of purposes. For example, such purposes can include identification, authentication, authorization, permissions, and synchronization priorities.

The computer readable medium 120, according to the present example, can be communicatively coupled with a reader/writer device (not shown) that is communicatively coupled via the bus architecture 208 with the at least one processor 102. The instructions 107, which can include instructions, configuration parameters, and data, may be stored in the computer readable medium 120, the main memory 104, the persistent memory 106, and in the processor's internal memory such as cache memory and registers, as shown.

The information processing system 100 includes a user interface 110 that comprises a user output interface 112 and user input interface 114. Examples of elements of the user output interface 112 can include a display, a speaker, one or more indicator lights, one or more transducers that generate audible indicators, and a haptic signal generator. Examples of elements of the user input interface 114 can include a keyboard, a keypad, a mouse, a track pad, a touch pad, a microphone that receives audio signals, a camera, a video camera, or a scanner that scans images. In some embodiments, the user input interface can include fitness trackers or other types of health monitoring devices. The received audio signals or scanned images, for example, can be converted to electronic digital representation and stored in memory, and optionally can be used with corresponding voice or image recognition software executed by the processor 102 to receive user input data and commands, or to receive test data for example.

A network interface device 116 is communicatively coupled with the at least one processor 102 and provides a communication interface for the information processing system 100 to communicate via one or more networks 108. The networks 108 can include wired and wireless networks, and can be any of local area networks, wide area networks, or a combination of such networks. For example, wide area networks including the internet and the web can inter-communicate the information processing system 100 with other one or more information processing systems that may be locally, or remotely, located relative to the information processing system 100. It should be noted that mobile communications devices, such as mobile phones, Smart phones, tablet computers, lap top computers, and the like, which are capable of at least one of wired and/or wireless communication, are also examples of information processing systems within the scope of the present disclosure. The network interface device 116 can provide a communication interface for the information processing system 100 to access the at least one database 117 according to various embodiments of the disclosure.

The instructions 107, according to the present example, can include instructions for monitoring, instructions for analyzing, instructions for retrieving and sending information and related configuration parameters and data. It should be noted that any portion of the instructions 107 can be stored in a centralized information processing system or can be stored in a distributed information processing system, i.e., with portions of the system distributed and communicatively coupled together over one or more communication links or networks.

FIGS. 1-18 illustrate examples of methods or process flows, according to various embodiments of the present disclosure, which can operate in conjunction with the information processing system 100 of FIG. 19.

What is claimed is:

1. A patient-centric universal health recording and payment system, comprising:
a synchronized server having synchronized patient information accessible and controllable by a patient;
a plurality of electronic medical record databases selectively synchronized with the synchronized server;
a universal user interface coupled to the synchronized server for presenting the synchronized patient information including information from all accessed providers;
a plurality of third party inputs to the synchronized server selectively providing information regarding the patient with respect to one or more among telemedicine information, nutrition intake information, and genetics information;
a plurality of patient sourced inputs to the synchronized server selectively providing information comprising one or more among patient monitored data, patient record outcome measures, and caretaker record outcome measures; and
a patient input defining accessibility among and between the plurality of electronic medical record databases, a source of the plurality of third party inputs, and a source of the plurality of patient sourced inputs.

2. The system of claim 1, wherein the system further comprises a patient health card providing patient authentication or patient identification as a condition precedent to enabling the patient input defining accessibility.

3. The system of claim 1, wherein the system further comprises a patient health card providing patient authentication or patient identification as a condition precedent to enabling the patient input defining accessibility and wherein the patient health card further provides funding for payments of health care services.

4. The system of claim 1, wherein the system further comprises a patient health card providing patient authentication or patient identification as a condition precedent to enabling the patient input defining accessibility and wherein the patient health card further provides funding for payments using cryptocurrency.

5. The system of claim 1, wherein the plurality of third party inputs comprises one or more among third party telemedicine, third party medical algorithms, third party medical treatment advice, third party disease and health management services, third party provided genetic sequencing information, and third party nutritional intake information.

6. The system of claim 1, wherein the plurality of patient sourced inputs comprises one or more among data obtained from passive remote data collection from the patient, remote diagnostics from the patient, wearables from the patient, fitness trackers from the patient, self recorded inputs from the patient, and caretaker inputs observed about the patient.

7. The system of claim 1, wherein the plurality of electronic medical record (EMR) databases comprises one or more among a primary care physician EMR, a hospital EMR, a specialist EMR and a skilled nursing facility EMR.

8. The system of claim 1, further comprising an Electronic Data Capture input to the universal user interface or the synchronized server enabling access to patient information for clinical research.

9. The system of claim 3, wherein the health card enables transactions and transfers of value among a patient account and a service provider.

10. The system of claim 3, wherein the health card enables transactions and transfers of value among a patient account, a service provider, and an insurance provider.

11. The system of claim 3, wherein the system further comprises a patient client device that couples with the patient health card and with the synchronized server.

12. The system of claim 1, wherein the user interface comprises a listing of a circle of trust comprising one or more among physicians, insurers, care providers, family, and friends which the patient can selectively grant access to all or a portion of the patient's medical information.

13. A system for a patient-centric universal health recording and payment, comprising:
a memory having computer instructions stored therein;
one or more processors coupled to the memory, wherein the one or more processors upon execution of the computer instructions cause the one or more processors to perform the operations comprising:
receiving a signal authenticating an authorized user using information at least in part from the memory, wherein the signal authenticating the authorized user is initiated by coupling a health card with a client device;
synchronizing a synchronization server in response to receiving a signal to instruct the synchronization server to selectively synchronize patient information from a plurality of sources including a plurality of electronic medical record databases;
providing a universal user interface coupled to the synchronization server for presenting the synchronized patient information including information from all accessed providers.

14. The system of claim 13, wherein the synchronizing of the synchronization server is in response to the health card coupling with the client device and the signal authenticating the authorized user.

15. The system of claim 13, wherein the one or more processors are configured to receive a plurality of third party inputs to the synchronization server selectively providing information regarding the patient and further configured to receive a patient input defining accessibility among and between the plurality of electronic medical record databases and a source of the plurality of third party inputs.

16. The system of claim 13, wherein the one or more processors are configured to receive a plurality of third party inputs to the synchronization server selectively providing information regarding the patient, to receive a plurality of patient sourced inputs to the synchronized server, and further configured to receive a patient input defining accessibility among and between the plurality of electronic medical record databases, a source of the plurality of third party inputs, and a source of the plurality of patient sourced inputs.

17. The system of claim 13, wherein the one or more processors are further configured to process a payment transaction using crypto currency initiated by use of the health card coupled to the client device and wherein the transaction is stored using a directed acyclic graph (DAG).

18. The system of claim 13, wherein the universal user interface comprises a listing of a circle of trust comprising one or more among physicians, insurers, care providers, family, and friends which the patient can selectively grant access to all or a portion of the patient's medical information.

19. A computerized method, the method comprising:
receiving a signal authenticating an authorized user using information at least in part from a memory stored on a health card that interfaces with a client device, wherein the signal authenticating the authorized user is initiated by coupling the health card with the client device;
synchronizing a synchronization server in response to receiving a signal to instruct the synchronization server to selectively synchronize patient information from a plurality of sources including a plurality of electronic medical record databases;
providing a universal user interface coupled to the synchronization server for presenting the synchronized patient information including information from all accessed providers in response to the signal to instruct the synchronization server to selectively synchronize.

20. The computerized method of claim 19, wherein the method further comprises:
receiving a plurality of third party inputs to the synchronization server selectively providing information regarding the patient;
receiving a plurality of patient sourced inputs to the synchronized server; and
receiving a patient input defining accessibility among and between the plurality of electronic medical record databases, a source of the plurality of third party inputs, and a source of the plurality of patient sourced inputs.
